(19) United States
(12) Patent Application Publication
(10) Pub. No.: US 2023/0295566 A1
(43) Pub. Date: Sep. 21, 2023

(54) METHOD FOR PRODUCING TUMOR-INFILTRATING T-LYMPHOCYTES (TIL) AND THEIR USE AS CELLULAR THERAPEUTICS FOR THE TREATMENT OF HUMAN TUMORS

(71) Applicant: ZELLWERK GMBH, Oberkramer (DE)

(72) Inventors: Hans HOFFMEISTER, Oberkramer (DE); Elke JAGER, Oberkramer (DE); Julia KARBACH, Oberkramer (DE); Evgeni SINELNIKOV, Oberkramer (DE); Dirk GUSTAVUS, Oberkramer (DE)

(21) Appl. No.: 18/041,487

(22) PCT Filed: Dec. 18, 2021
(86) PCT No.: PCT/DE2021/000194

§ 371 Date: Feb. 13, 2023

(30) Foreign Application Priority Data
May 27, 2021 (DE) 10 2021 002 748.5

(51) Int. Cl. C12N 5/0783

(52) U.S. Cl. CPC C12N 5/0636; C12N 2500/02; C12N 2501/2302; C12N 2501/2312; C12N 2501/599

(57) ABSTRACT

The invention relates to a method for isolating, activating and propagating immune cells, more particularly tumor-infiltrating autologous T-lymphocytes (TIL) from primary tumor tissue, metastatic growths, lymphatic tissue but also T cells from other tissues (e.g., blood, lymphatic fluid) in a meander perfusion bioreactor and to the production of immune cellular therapeutics therefrom for controlling tumors of the pancreas, lung, liver, prostate, breast, ovaries, stomach, colon, rectum, bones, brain, skin and other malignant tumors.

Process for the production of tumor-infiltrated T-lymphocytes (TIL) and their use as cell therapeutics for the treatment of human tumors

The invention relates to a method for isolating, activating and multiplying immune cells, in particular tumor-infiltrated autologous T lymphocytes (TIL) from primary tumor tissue, metastases, lymphatic tissue, but also T cells from other tissues (e.g. B. blood, lymph fluid) in a meander perfusion bioreactor and the production of immune cell therapeutics from it to combat tumors of the pancreas, lungs, liver, prostate, breast, ovary, stomach, colon, rectum, bone, brain, the skin and other malignant tumors.

From the prior art, for the cultivation of lymphocytes and the production of immune cell therapies from them as “Advanced Investigational Medicinal Products” (AIMP) for the treatment of cancer diseases, the use of the following interleukins together with the following antibodies, growth factors with coating products, such as plasma expanders gelatin and supplements such as polyethylenimine; Polyethylene glycol, prostacyclin complement substances known as C1 to C9:
> Interleukin 2
   with the Anti CD 3 antibody
   with the antibodies anti CD 3 and anti CD 16,
   with the antibodies anti CD 3 and anti CD 28,
   with the antibody anti CD 16,
   with the anti-CD 28 antibody,
   with the antibodies anti CD 28 and anti CD 56,
   in connection with interleukin 7 and interleukin 17,
   in connection with interleukin 12 and the antibody CD 3,
> Interleukin 1a in combination with Interleukin 15 and the Anti CD 28 antibody,
> Interleukin 10 with the antibody Anti CD 8,
> Interleukin 21 with the antibody anti CD 56,
> Interleukin 19 with the Anti CD 3 antibody.

In addition, the use of interleukin 21 with the antibody 4-1 BBL for an in vitro amplification method for efficient and highly cytotoxic natural killer (NK) cells is known from the prior art.

Furthermore, in the prior art (J. Immunol.2004; 172: 4779) Experimental results discussed the effects of a combined IL-12 gene transfer with 4-1 BB costimulation on the cooperative anti-tumor effect against a model tumor (lung metastasis model).

IL-12 gene transfer is combined with 4-1 BB costimulation to investigate an already mentioned cooperative anti-tumor effect against this model tumor.

It has been hypothesized that the innate immune response mediated by IL-12-activated natural killer (NK) cells triggers activation of the immune system and leads to activation of T cells, while 4-1 BB costimulation enhances the function of Improved tumor-specific T cells.

In contrast, neither IL-12 gene transfer nor administration of anti-4-1 BB antibodies are effective alone.

The combination therapy significantly delayed the growth of subcutaneously inoculated tumors and 50% of the tumor-bearing mice survived with complete tumor regression.

In Gene Ther.2005 Oct; 12(20): 1526-33 put Xu DP, Sauter BV, Huang TG,

Meseck M, Woo SL, Chen SH. demonstrate that systemic delivery of Ig-4-1 BBL can generate a better antitumor response than local gene delivery. Ig-4-1 BBL had equivalent biological functions compared to the agonistic anti-4-1 BB antibody.

Thus, soluble 4-1 BBL dimmer can be developed as a promising agent for human cancer therapy.

Only the effects of therapy with the above combinations on mouse carcinomas are discussed here and no method, in particular no culture medium, for isolating and multiplying cells from tissue parts of tumors, metastases and other tissues is described.

Furthermore, all cell cultivation methods known from the prior art have a supply of oxygen to the cells in the medium from a supernatant or overflowing overlay atmosphere or from a supernatant and below overlay and underlay atmosphere, with the underlay atmosphere being supplied by the supernatant medium with contained therein cells releases additional oxygen to the supernatant medium by means of an oxygen-permeable membrane.

A device or possibility integrated into the cultivation process that ensures the growing oxygen requirement of the multiplying immune cells during a cultivation run is not provided for in the cultivation process described and is also not feasible (e.g. B. GRex vessels; Aastrom Vericell system).

It is also not known whether the positive effect of the combined use in the mouse model arises in humans in the same way.

The effects found in the mouse model are very often not found in humans.

WO 2015 189356 A1 relates to a composition for expanding lymphocytes, comprising at least two types of cytokines selected from interleukin 2 (IL-2), interleukin 15 (IL-15) and interleukin 21 (IL-21).

It also relates to a method for producing a population of clinically relevant lymphocytes, comprising the steps of: obtaining a body sample from a mammal, in particular a tissue sample or body fluid sample, comprising at least one lymphocyte and optionally separating the cells in the body sample, culturing the body sample in vitro to to expand and/or stimulate lymphocytes in the sample, wherein the culturing comprises using IL-2, IL-15 and/or IL-21 and optionally determining the presence of clinically relevant lymphocytes in the cultured sample.

The present invention also relates to immunotherapy and the population of clinically relevant lymphocytes.

The body sample is selected from peripheral blood of a mammal, in particular a human is selected with a tumor disease or a mammal at risk of developing a tumor disease or with an infectious disease or at risk of developing an infectious disease or with an autoimmune disease or at risk of Development of an autoimmune disease. WO 2015 189357 A1 describes a composition for expanding lymphocytes, comprising at least two types of cytokines selected from interleukin 2 (IL-2), interleukin 15 (IL-15) and interleukin 21 (IL-21).

It also relates to a method for producing a population of clinically relevant lymphocytes, comprising the steps of: obtaining a body sample from a mammal, in particular a tissue sample or body fluid sample, comprising at least one lymphocyte and optionally separating the cells in the body sample, culturing the body sample in vitro to expand and/or stimulate lymphocytes in the sample, the culturing involving the use of IL-2, IL-15 and/or IL -21 and optionally determining the presence of clinically relevant lymphocytes in the cultured sample. The present invention also relates to immunotherapy and the population of clinically relevant lymphocytes

WO 2020025706 A1 describes a method for producing a T cell product containing tumor-overreactive immune cells (TURICs) and a composition containing at least one T cell product containing TURICs for use in treating a cancer patient.

The method comprises the steps of a) providing a body sample containing T cells from a patient; b) optionally isolating the T cells from the body sample; c) stimulating the T cells in vitro in the presence of a cytokine cocktail of the cytokines interleukin 2 (IL-2), interleukin 15 (IL-15) and interleukin 21 (IL-21) and a stimulating peptide or group of stimulating peptides ; d) determining a reactivity factor in the T cell sample, the reactivity factor being indicative of the presence of T cells that target the stimulatory peptide or at least one peptide from the group of stimulatory peptides; e) if the reactivity factor is positive, identifying the T cell sample as a tumor reactive T cell sample; otherwise identifying the T cell sample as a non-reactive T cell sample; f) culturing the non-reactive sample in vitro in the presence of the cytokine cocktail of IL-2, IL-15 and IL-21 and either one of autologous tumor cells or the stimulating peptide or group of stimulating peptides, g) optionally stimulating the T - cell product in vitro in the presence of the cytokine cocktail of IL-2, IL-15 and IL-21 and the stimulating peptide or group of stimulating peptides; h) determining the reactivity factor in the T cell product; and i) if the reactivity factor is positive, the T cell product is selected as a TURIC-containing T cell product.

WO 2020 198031 A1 discloses a method and the production and use of lung cancer-specific marrow-infiltrating lymphocytes (“MILs”). The method comprises the steps: a. culturing a bone marrow sample obtained from the lung cancer patient with an anti-CD3 antibody and an anti-CD28 antibody in a hypoxic environment to produce hypoxic activated marrow-infiltrating lymphocytes; b. culturing the hypoxically activated marrow-infiltrating lymphocytes in a normoxic environment to produce the therapeutically activated marrow-infiltrating lymphocytes; and (c) administering the therapeutically activated marrow-infiltrating lymphocytes to the subject with lung cancer.

The invention according to EP 3730608 A1 relates to a method of treating a patient with cancer, the method comprising administering expanded tumor-infiltrating lymphocytes (TILs), comprising: a) obtaining
a) first population of TILs from a tumor derived from a patient was resected by processing a tumor sample obtained from the patient into multiple tumor fragments;
b) adding the tumor fragments into a closed system;
c) performing a first expansion by culturing the first population of TILs in a cell culture medium comprising IL-2 to produce a second population of TILs, the first expansion being performed in a closed container providing a first gas-permeable surface, wherein the first expansion is performed for about 3 to 14 days to obtain the second population of TILs, wherein the second population of TILs is at least 50-fold larger than the first population of TILs, and wherein the transition from step (b) to step (c) takes place without opening the system;
d) performing a second expansion by supplementing the cell culture medium of the second population of TILs with additional IL-2, OKT-3 and antigen presenting cells (APCs) to create a third population of TILs, the second expansion being performed for about 7 to 14 days to the third population of TILs, wherein the third population of TILs is a therapeutic population of TILs comprising an increased subpopulation of effector T cells and/or central memory T cells compared to the second population of TILs, wherein the second expansion is carried out in a closed container providing a second gas permeable surface and wherein the transition from step (c) to step (d) occurs without opening the system;
e) harvesting the therapeutic population of TILs obtained from step (d), transitioning from step (d) to step (e) without opening the system; and
f) transferring the harvested TIL population of step (e) into an infusion bag, wherein the transferring from step (e) to (f) occurs without opening the system;
g) cryopreserving the infusion bag comprising the harvested TIL population of step (f) using a cryopreservation method; and
h) population of TILs from the infusion bag in step (g) to the subject

The number of TILs sufficient to administer a therapeutically effective dosage in step (h) is about 1×10⁹ to about 9×10¹⁰.

The pharmaceutical composition is used for the manufacture of a medicament for the treatment of cancer, which cancer is selected from the group consisting of melanoma (including metastatic melanoma), ovarian cancer, cervical cancer, non-small cell lung cancer (NSCLC), lung cancer, bladder cancer, breast cancer, human cancer papillomavirus, head and neck cancer (including head and neck squamous cell carcinoma (HNSCC)), kidney cancer and renal cell carcinoma.

WO 2020231 058 A1 relates to activated lymphocytes comprising cytokine-induced killer cells in which CD8 + CD56 + NKG2D + cells are present in a proportion of 20% or more and a production method therefor and in particular to activated lymphocytes which cytokine-induced Killing includes cells which have high tumor cell-killing ability and growth rates and are almost free from side effects because they do not require the combined administration of interleukin-2 and a manufacturing method thereof.

EP 3565888 A1 discloses a method for expanding tumor-infiltrating lymphocytes as a two-stage/three-stage method using the cytokine IL 2 with and anti-CD3 (Okt3)/anti-CD28 antibodies.

The cell culture medium contains IL-2, OKT-3 (anti-CD3) antibody, peripheral blood mononuclear cells (PBMCs) and optionally the TNFRSF agonist such as I-4bb and a second TNFRSF agonist.

The culture media described in the prior art are also all very expensive.

This makes it difficult to conduct statistically meaningful clinical studies and stands in the way of a general application of therapy with immune cells.

The object of the invention is to develop a method for cultivating cells, in particular tumor-infiltrated T-lymphocytes (TIL) and other T-cells from human lymphoid tissues in a cultivation system with meander perfusion bioreactors, with which existing problems such as low standardization and reproducibility, difficult processes for the mass production of these cells, further complex and inconvenient conditions in general clinical use as well as high production costs, which exist in the conventional in vitro culture methods, are radically solved.

Surprisingly, it was found that the object of the invention is achieved by a method in which
a defined number of comminuted pieces of tissue from a patient’s individual tumor are introduced into a meander perfusion bioreactor and distributed evenly,
the cells growing out of the tissue pieces, in particular tumour-infiltrated T-lymphocytes, sediment at the bottom of the clot,
After sedimentation, the cells form a largely stationary cell layer with close and at the same time alternating contact between the cells for good proliferation, in which they touch, but slight movements also occur, with TIL cell counts in the sedimented layer at 0.1 to 2 × 106TIL/cm2, preferably 0.5 to 1× 1 0⁶TIL/cm2 for steady growth,
a meandering guidance of the medium flow through a channel of a perfusion bioreactor with an overflow according to a Froud number <0.005, preferably <0.002 and a bottom flow according to a Froud number of 0 or close to 0 occurs, whereby the medium flows in a directed laminar flow over the sedimented TIL, similar to the blood flow in natural blood vessels and does not cause cell stress,
the medium consists of a conventional basic medium supplemented with AB human serum, cytokines, antibodies and irradiated and disintegrated human feeder cells, the cytokines consisting of interleukin 12 alone or of a mixture of interleukin 2 and interleukin 12 or of a mixture of IL12, IL15 and all mixtures additionally contain the antibody 4-1 BB,
the pieces of tissue and the cells growing from them in the bioreactor vessels from the underlay chamber and the overlay chamber are simultaneously supplied with oxygen in a hyperoxic or normoxic manner.

The combinations mentioned above are necessary in order to ensure a specific proliferation of mutated TILs in the initial phase, but also mutated T cells from other tissues (e.g. B. blood, lymphatic fluid) in such a way that cells primed by neoantigens of the tumor are proliferated, whereby in the later phase of proliferation the ability of the TIL to divide is retained considerably longer (and thus significantly higher yields are achieved) compared to the same TIL that are cultivated in static culture or in turbulent flowing medium

The new method enables the production of multiple 10⁹ to multiple 10¹⁰ immune cells as ATMP in a completely closed cultivation process.

For this purpose, a single-use perfusion bioreactor is initially installed and operated in the sterile room of our GMP breeder and monitored by a control unit: The supply of medium and gases is regulated; pH value, pO2 concentration and temperature are continuously measured in the medium by sensors and kept constant.

As the amount of TIL in the bioreactor vessel increases, the supply of medium is automatically increased via an algorithm.

If sufficient tumor tissue is available, a 150 MM bioreactor is used.

The shredded pieces of tissue containing immune cells are placed in the sterile room of the GMP Breeder in the single-use bioreactor, which is connected and ready for operation.

The approximately 1 mm3 large pieces of tumor tissue are spread evenly in the Medium of the overlay chamber distributed.

In the bioreactor vessel, TIL grow in a standard medium that is supplemented with human serum, IL2, Oct3 and irradiated feeder cells and/or also with irradiated and then ultrasonically disintegrated feeder cells from the tumor tissue pieces in 7 to 14 days and multiply continuously .

This way 4 to 10 x 10⁹ TIL can be grown.

During the cultivation, the medium is circulated at a low flow rate over the sedimented TIL, a defined portion of the medium is increasingly replaced by fresh medium, depending on the glucose consumption.

This is automatically controlled by an algorithm in the control unit.

In the case of the TIL, close and at the same time alternating contact between the cells is necessary for good proliferation during the entire expansion period.

A proliferation of the TIL presupposes that the corresponding cells form a largely stationary cell layer after sedimentation, in which they touch each other, but slight movements also occur, with cell counts of 0.1 to 2 × 10⁶TIL/cm2 in the sedimented layer of the TIL, preferably 0.5 to 1× 10⁶TIL/cm² occur and there is a steady increase in TIL.

If larger amounts of TILs are to be administered several times for the treatment of a patient, also at intervals, the amount of TIL harvested from the first cultivation run can be split and used as a working cell bank for the parallel colonization of 4 to 8 bioreactors 150 MM .

Multiple 10L10 TIL can be generated with these bioreactor runs.

TILs are cultivated when only a small amount of tumor tissue is available (e.g. B. from biopsies), a meander perfusion bioreactor 30 MM with overlay and underlay chamber used.

If the TIL obtained from an expansion run in a 30 MM bioreactor is to be increased further, this is possible in the following way: A 150 MM bioreactor is set up ready for operation in the GMP Breeder and a 30 MM bioreactor is also placed on top of it. The 30 MM bioreactor is connected to the 150 MM bioreactor underneath via an initially closed hose connection.

As soon as the TIL in the 30 MM bioreactor have increased to the usual amount and the exponential growth levels off, the TIL are brought into suspension by shaking them briefly, the hose connection to the 150 MM bioreactor is opened, the TIL suspension is discharged through the outlet nozzle with sieve mesh and the connecting hose into the bioreactor vessel 150 MM transferred. The supply of gases, automatically adjusted medium supply as well as control and documentation of the cultivation process are continued. After 3 to 7 days of expansion in the 150 MM bioreactor, 4 × 10⁹ to 1 × 10L10 TIL can be harvested, which are then suitable for further expansion runs.

The directed laminar overflow of the sedimented TIL with medium, with homogeneous concentrations of glucose and lactate being maintained over the entire expansion period, and the stable oxygen partial pressure in the medium ensure high reproducibility of the TIL produced by the methods described above. In addition, it has been shown that certain subpopulations can be preferentially increased with the perfusion technology.

The proportions of some sub-populations of the expanded TIL can be influenced by varying the concentrations of IL2, Oct3 and IL12 as well as the duration, chronological sequence and subsequent washing out of these additives by supplying fresh medium without these supplements. So e.g. For example, the proportion of CD8+TIL can be significantly increased by short exposure to IL12. Furthermore, the supplementation with small amounts of polyethylene glycol or polyethylene imine in the later course of the expansion phase in the bioreactor leads to qualitatively and quantitatively improved expression of trafficking receptors in the harvested TIL. With the supplementation described above, an enriched amount of neoantigen-primed TIL can be generated that reach the tumor/tumor metastases.

The immune cells obtained with the new cultivation method offer further advantages in the production of ATMP to fight cancer compared to the previously known methods in that the method for the production of cell therapeutics (approved by the responsible authorities for the parallel cultivation of cells from different Patients in the same clean room because the bioreactors, which are closed anyway, are each also operated in the GMP breeder, which is a very effective sterile bank.

This prevents cross-contamination between patient samples.

This cultivation method is also of particular importance due to its modular design as a tabletop device.

The production of patient-specific TIL (or other immune cell or stem cell therapeutics) is in accordance equipped clinical research facilities and facilitates the cultivation of cells close to the patient under GMP conditions.

With the method according to the invention, the cultivation and isolation of sufficient amounts of TIL, which have a cytotoxic effect on the mutated tumor cells, is reproducible in a controlled process and routine production for use in a cell therapy is feasible.

The method is designed as a two-stage cultivation method for the ex-vivo isolation, activation and propagation of TIL from tissue parts of tumors, metastases and other tissues, with the cells being transplanted from comminuted tissue parts into the medium located in the perfusion bioreactor vessel begin to grow.

After a certain density of mature TIL has been reached, it is specifically activated with anti-IL2 for a further short period of time and then expanded further in medium without anti-IL2.

Harvest of the TIL from an initial cultivation run in a perfusion bioreactor is initiated when the bioreactor has reached full growth, as evidenced by the leveling off of the previously exponential increase in glucose in the culture medium.

For the GMP-compliant TIL harvest, the bioreactor is briefly shaken.

The TIL suspension is transferred through the sieve port of the bioreactor via a sterile connected tube into a 200 ml blood bag or 100 ml blood bag. The blood bag is separated from the bioreactor and the number of TIL in the blood bag is determined in an aliquot. Depending on the tumor tissue, 4 to 10 x 10⁹ TIL can be expected from a 150 MM perfusion bioreactor.

The suspension in the blood bag from the first cultivation run is used as the working cell bank.

Volume fractions each containing 0.75 x 10⁹ TIL are withdrawn. One or more of the partial suspensions are immediately transferred to a 150 MM bioreactor and immediately further expanded there in further cultivation runs in order to have a first dose of TIL available for the patient as early as possible. The remaining volume fractions from the first TIL harvest are stored cryopreserved in suitable cryo-bags with the addition of DMSO and then multiplied in a second cultivation step in 150 MM bioreactors if signs of the tumor disease are still detected after the first TIL treatment and e.g. B. a higher dosage is considered medically necessary or recurrences or metastases make this necessary.

The second expansion of a patient-specific TIL ATMP takes place in a standard medium supplemented with IL2, IL12 and 4-1 BB-AB.

Yields of TIL are usually similar to those in the first proliferation run.

The TIL suspensions harvested in the second cultivation run are processed as follows: A 150 MM bioreactor contains a TIL suspension with a volume of 50 ml.

The suspension is transferred through the sieve into a blood bag with a volume of 100 ml and stored motionless for 3 hours at 2° to 8° C. in such a way that the TIL in the bag can sediment opposite the two hose connections of the bag. The medium supernatant is siphoned off sterilely from the blood bag leaving a residual volume of about 20 ml. The TIL are then resuspended in 80 ml of 0.9% NaCl solution, filled into centrifuge tubes (50 ml), centrifuged and then resuspended again in a total of 90 ml of 0.9% NaCl solution containing 2% human albumin and Contains 10% DMSO. For the cell count and the analyzes for the release of the ATMP, 10 ml of the suspension are taken, distributed in cryotubes and subjected to the necessary analyses. The cell count is determined in samples taken (10 ml in total) and FACS analyzes are carried out. The remaining 80 mL of TIL suspension should contain 3 to 8 x 10⁹ TIL. The suspension is divided into additional 100 ml blood bags so that each bag contains 2.5 x 10⁹ TIL in a 0.9% NaCl solution with 2% human albumin.

Once the TIL-AIMP bags have been released on the basis of the pharmacopoeia specifications, the preparations are transported and handed over to the doctor treating the patient under the known conditions and within the known deadlines. The cell suspension must be used for infusion within 6 to a maximum of 20 hours. When an IV bag is at room temperature, it is infused into the patient by vein. The dose is determined by the attending physician.

In one interpretation of the inventive method, the ex vivo multiplied TIL can be filtered from the tissue residues from a fully grown bioreactor 150 MM, harvested, washed, centrifuged, resuspended in NaCl solution with human albumin as defined portions in infusion bags and under controlled transport conditions within fewer hours to the patient transported there, brought to room temperature and infused into the patient to treat one of the tumor diseases mentioned above (cancer of the pancreas, lungs, endometrium, breast, colon, liver, brain, prostate, stomach, melanoma, advanced stage lymphomas) for which no other therapy is known.

In a further embodiment of the invention, the tumor-infiltrated lymphocytes (TIL) or T-cells of other origin are cultivated as a pharmaceutical composition as a cell therapeutic, the cell therapeutic being cultivated for the treatment of or a combination thereof.

For clinical applications, cryopreserved vials are thawed in such a way that, in a second expansion stage, therapeutically useful amounts of TIL are produced on schedule.

For this purpose, after washing out the freezing medium, the TIL are resuspended in a suitable culture medium.

The TIL suspension (50 to 100 million vital cells) is transferred to another meander bioreactor that is ready for operation. This second process step for the further expansion of TIL is carried out in a meander bioreactor, which is similar to the meander bioreactor described above, but which has a larger colonization area. Freshly supplemented culture medium is used for further propagation. After 12 to 20 days of expansion, more than 500 million cells regularly grow in the meander perfusion bioreactor. TIL high. The TIL are harvested, washed and resuspended in NaCl solution with human albumin. The TIL suspension is filled into an infusion bag. Viability and cell number are determined on the day of cell harvest and further analyzed according to the specifications of the pharmacopoeia sterility, marker profile, paracrine production of the cells. The TIL suspension is kept at room temperature and immediately transported to the clinical partner. The TIL must be applied after 12 to 20 hours at the latest.

Excess amounts of TIL are cryopreserved.

They are available for the production of further doses of TIL that will be necessary later, for 500 million TIL max. 100 ml NaCl solution with human albumin are used.

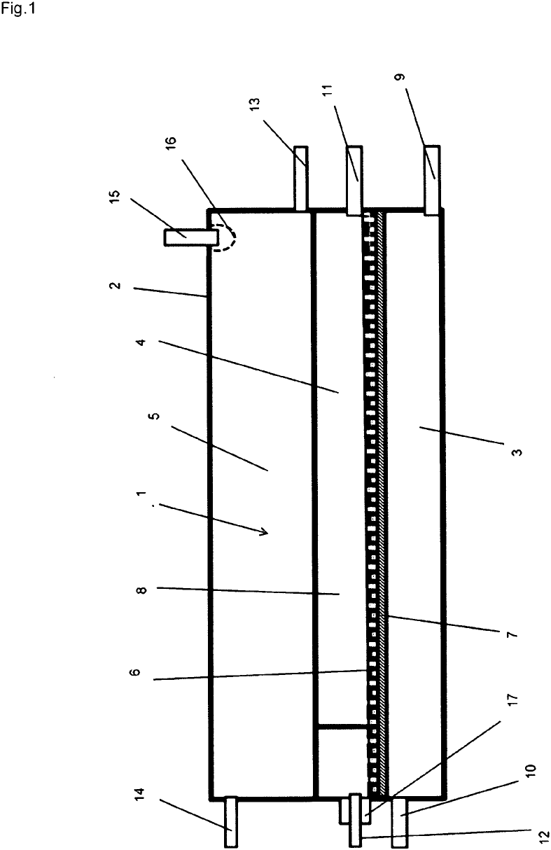

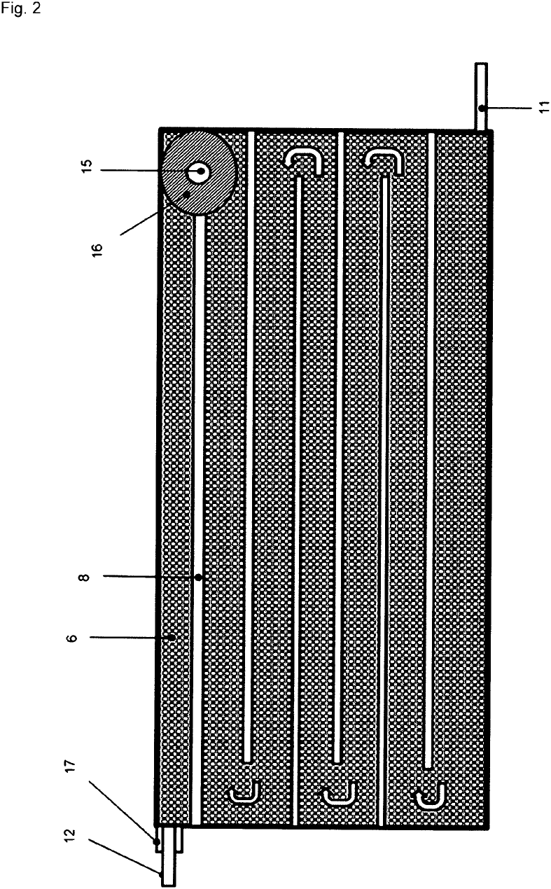

The invention is explained in more detail using an example for the isolation and propagation of TIL in a meander perfusion bioreactor, 1 shows a schematic sectional view of the meander bioreactor and FIG. 2 shows a schematic plan view of the meander bioreactor in which the method according to example 1 is carried out and
1 bioreactor vessel
2 lid
3 Unterlay chamber
4 Meander perfusion chamber
5 overlay chamber
6 bottom plate
7 foil
8 partition wall
9 Feeding underlay atmosphere]
10 Dissipation of underlay atmosphere
11 feeding culture medium
12 Removal of culture medium
13 Feeding overlay atmosphere
14 Dissipation overlay atmosphere
15 outlet nozzle
16 mesh
17 Mean overflow for cell broth.

### EXAMPLE 1

Starting material for the production of TIL or other T-cells are parts of tissue, which z. B. during the surgical removal of solid organ tumors or their metastases or their immediately surrounding tissue or removed for this purpose.

A tissue volume of about 1 ml is usually sufficient as starting material.

The tissue samples are placed in aTransport vessel spent with medium.

The vessel is kept sealed at room temperature and transported from the tissue removal site to the clean room area for the production of the immune cell preparations.

The pieces of OP tissue are comminuted in a clean room under an LFB into pieces of 1 to 2 mm3 in size and, in the first stage, transferred to a meander perfusion bioreactor in accordance with DE102018000561.6.

The shredded pieces of tissue are evenly distributed in the meander perfusion bioreactor and cultivated in the perfusion mode.

The meander bioreactor is connected ready for operation in a GMP breeder and is continuously monitored by the control unit, largely automatically controlled and documented.

The meander perfusion bioreactor consists of a rectangular bioreactor vessel 1 made of preferably clear polymer material, which is sealed with a cover 2 in a sterile manner.

The bioreactor vessel 1 is divided into three chambers 3, 4, 5 arranged one above the other, with the lowest chamber 3 as an underlay chamber, the chamber 4 arranged above the underlay chamber 3 as a meander perfusion chamber and the one above the meander perfusion Chamber 4 arranged chamber 5 are designed as an overlay chamber.

The underlay chamber 3 and the meander perfusion chamber 4 are separated from one another by a perforated base plate 6 with a film 7 which is tightly fastened thereon and is permeable to oxygen.

The underlay chamber 3 has inlets and outlets 9, 10 which allow gases to flow through, in particular mixtures of air and oxygen or nitrogen and oxygen, in which the proportion of oxygen is regulated.

The oxygen diffuses preferentially to nitrogen through the gas-permeable film 7, which is tightly attached to the perforated base plate 6 of the meander perfusion chamber 4.

During the flow through the meander perfusion chamber 4, controlled amounts of oxygen enter the culture medium during the expansion of the cells both from the overlay and from the underlay chamber (through diffusion).

The meander perfusion chamber 4 is provided with inlets and outlets for culture media 11, 12, and there are also inlets and outlets 13,14 for the overlay atmosphere and an outlet connector 15 in the overlay chamber 5.

A screen fabric 16 is arranged on the outlet socket 15 inside the overlay chamber 5 in front of the outlet.At the outlet 12 of the meander perfusion chamber 4 there is also an overflow 17 for discharging the used cell broth. The overflow is adjustable in height.

The meander perfusion chamber 4 consists of the base plate 6) with strip-shaped partitions 8 arranged on the upper side 8, dividing the base plate 6 and forcing a meandering flow through the meander perfusion chamber 4 with gases and medium, with the distance A of the partitions 8 from each other as well as from the side and end walls of the meander perfusion chamber 4 is chosen such that an averaged laminar overflow with a Froude number <0.005 forms in the stream thread, in the channel formed by the partition walls 8 .

As the number of cells increases as the number of cells (TIL or other cells) multiplies, the supply of fresh medium is continuously increased, with this being automatically controlled by a corresponding algorithm. The bottom flow remains close to a Froude number of 0, which prevents the cells from being stirred up. The flow conditions described prevent cell stress and ensure a homogeneous supply of nutrients over the entire cell layer growing at the bottom of the channel.

The overlay chamber (5) has inlets and outlets 13,14 for the overflow of gases.

The overlay chamber is flown through with the same gas/gas mixture as the underlay chamber 5), whereby the cell is supplied with oxygen either hyperoxically up to 90% O2), normoxically (21% O2) or hypoxically (up to 2% O2), depending on the cell type will

In a cultivation run, cells usually grow out of the tissue pieces for 7 to 14 days, and the mature cells multiply at the same time.

In this way, TIL can be produced in larger quantities from tumor tissue.

But TINK and other cells can also be obtained from tissues in this way.

When the glucose consumption in the bioreactor reaches 100 to 300 mg per day, the TIL are separated from the tissue residues as filtrate via the outlet nozzle 15 of the bioreactor vessel 1 provided with sieve fabric 16 .

From the transfer of the tissue pieces into the bioreactor vessel 1 in the first stage, the entire process up to the filling of activated and expanded cells takes place in a completely closed vessel system.

Is cultivated in a suitable medium with a specific mixture of AB human serum, cytokines, antibodies and Irradiated human feeder cells is temporarily supplemented, with cytokines in the form of interleukin 12 or a mixture of interleukin 2 and interleukin 12 and the antibodies in the form of antibody 4-1 bb being used.

In some cases, faster propagation and activation can be achieved with supplements that are fixed to the surfaces of the bioreactor channel. In the case of TIL and other immune cells, the TIL multiplied and separated in this first stage are centrifuged, resuspended in freezing medium and aliquoted (around 50 million TIL per vial). The samples are stored in the gas phase over liquid nitrogen.

With the method according to the invention for the isolation and propagation of cells from tissue parts of tumors, metastases and other tissues, it is possible in a single closed process step to grow certain cells from small pieces of tissue, from autologous pieces of tissue, both in the culture medium located in the bioreactor and Simultaneously activate cells, multiply them and then separate them from tissue debris.

In particular, tumor-infiltrating autologous T lymphocytes (TIL) can be obtained in this way from tumor tissue, metastasis tissue, their surrounding tissue and from lymph nodes which are affected by tumor cells. Under the flow and gassing conditions in the channel of a perfusion bioreactor, the culture medium according to the invention leads to rapid growth of the cells. In the case of TIL, the cells that are particularly preferably expanded are those that have specific cytotoxicity compared to the tumor cells from which or from their environment they are obtained

1. A method for the ex-vivo extraction of TIL and other T cells and use as patient-specific cell therapeutics to combat tumors, in which a defined number of cells in a first stage of the process from comminuted tissue parts in a closed meander perfusion bioreactor vessel are introduced and distributed evenly; the cells grow into the culture medium located in a perfusion meander bioreactor, simultaneously activated and multiplied with a meandering guidance of the medium flow through a channel of the perfusion bioreactor with an overflow according to a Froud number <0.005, preferably, 0.002 and a bottom flow according to a Froud number of 0 or close to 0 and the medium flows in a directed laminar flow, which does not cause cell stress, over the sedimented TIL and, after reaching a certain density of fully grown and expanded cells is seperated from the tissue parts, in right harvested in this form, the cells separated in this way are centrifuged, resuspended in freezing medium in vials, aliquoted and cryopreserved and in a second step the cryopreserved vials are thawed, the freezing medium is washed out and the cells are resuspended in a suitable culture medium and placed in another operationally installed meander perfusion bioreactor with the same or larger settelment area in the ratio of 5 to 1 compared to the meander perfusion bioreactor of the first stage, are transferred with the culture medium being perfused in the bioreactor vessels of the first and second stages in the bioreactor vessels in a directed manner with oxygen and the culture medium is supplemented with a mixture of AB human serum, cytokines and antibodies characterized in that
• the cells growing out of the pieces of tissue at the bottom of the sedimentation of the channel of the meander perfusion bioreactor,
• The cells are largely dormant after sedimentation, form cell layer in which they touch, but also light ones movements occur, with TIL cell counts in the sedimented layer at 0.1 to 2 x, 106TIL/cm2, preferably 0.5 to 1 × 10⁶TIL/cm2 for steady proliferation
• the culture medium contains cytokines and antibodies, the cytokines being in the form of interleukin 12 or a mixture of interleukin 2 and interleukin 12 and the antibodies being in the form of antibody 4-1 bb and
• The cells receive a hyperoxic or normoxic supply of supplemented medium and oxygen depending on the increased amount of TIL or other T cells through an automatically controlled supply.

2. The method according to claim 1, characterized in that the cells are cultivated as tumor-infiltrated lymphocytes (TIL) or tumor-infiltrated NK cells (TINK), or other cell types in the meander perfusion bioreactor in appropriately supplemented media.

3. Method according to claim 1 characterized in that
• the cryopreserved vials are thawed for further expansion,
• the freezing medium is washed out by centrifuging and resuspending the cells in freshly supplemented medium containing a mixture of AB human serum, cytokines and antibodies.
• This cell suspension is transferred in a second stage into one or more meander perfusion bioreactors of the same design as in the first stage, but with a larger colonization area or is split
• the TIL in the bioreactors expands over 12 to 20 days after the cultivation is harvested, washed in NaCl solution and centrifuged,
• resuspended the cells in a solution containing 0.9% NaCl, 2% albumin and 10% contains DMSO, and this suspension is filled into defined portions in 100 ml cryobags after a conventional cooling process and stored over nitrogen, and
• the TIL suspensions thus stored are retrieved, thawed and dosed appropriately to the patient.

4. Process for the production of a cell therapy according to claim 2, characterized in that the cell therapy consists of tumor-infiltrated lymphocytes (TIL), as a pharmaceutical composition for use as a cell for the treatment of tumors and infections.

5. Process for the production of a cell therapy according to, claim 2, characterized in that the cell therapy for the treatment of pancreatic, pulmonary, ovarian, breast, large intestine, bone marrow, liver, cerbral prostate, -, gastric rectum blood, glioma, melanoma, lymphoma or a combination thereof.
